(19) United States
(12) Patent Application Publication
(10) Pub. No.: US 2007/0118138 A1
(43) Pub. Date: May 24, 2007

(54) ALIGNMENT AND CONNECTION DEVICE OF FEMUR CUTTER AND TIBIA CUTTER AND METHOD OF KNEE ARTHROPLASTY USING THE SAME

(76) Inventors: JAI-GON SEO, Seoul (KR); CHONG-BUM KIM, Goyang (KR)

Correspondence Address: PARK LAW FIRM, 3255 WILSHIRE BLVD, SUITE 1110, LOS ANGELES, CA, 90010, US

(21) Appl. No.: 11/163,674

(22) Filed: Oct. 26, 2005

(51) Int. Cl. A61F 5/00

(52) U.S. Cl. 606/87

(57) ABSTRACT

An alignment and connection device by which it is possible to follow the determined extension gap minutely and align the femur cutter and tibia cutter three-dimensionally is disclosed. This alignment and connection device comprises a fixation part in which a guide slot is formed in front center and a through hole for a locking pin to be inserted is formed on the side of the guide slot and an indicating line is marked thereon; a supporting part which is assembled slidably to the guide slot of the fixation part, and having a slider marked with specifications corresponding to the extension gap determined and a lug portion inserted into the cutting groove of the femur cutter; and a fixing means for fixing the supporting part to the fixation part.

### BACKGROUND OF THE INVENTION

1. Field of the Invention

The present invention relates to an alignment and connection device for aligning and connecting the femur cutter and tibia cutter in knee arthroplasty and a method of knee arthroplasty using the device. More particularly, the present invention relates to an alignment and connection device of femur cutter and tibia cutter by which it is possible to minutely follow the determined extension gap and three-dimensionally connect the alignment of femur cutter and tibia cutter, and a method of knee arthroplasty using the device.

2. Description of the Related Art

Knee arthroplasty has become one of the most commonly performed surgeries, in which a damaged or deformed knee joint due to congenital deformation, traumatic injuries, diseases, degenerative arthritis, etc. is removed and replaced with an artificial joint. In such a knee arthroplasty, accurate cutting of the damaged knee joint is very important to prevent side effects after surgery and prolong the life of the replaced artificial knee joint.

FIG. 1 is a view showing an ideal cutting of extension gap. As shown in the drawing, in order to cut an extension gap ideally, the femur and tibia should be aligned in agreement to a mechanical axis so that the bottom end cut surface of femur 11 and the top end cut surface of tibia 12 are maintained in parallel each other.

We will take a look at the conventional knee arthroplasty, in which, as shown in FIG. 2a, a femur cutter 21 is mounted on the bottom end of femur 11 using alignment device (not shown) and the bottom end of the femur 11 is independently cut through a cutting groove 21-1. Next, as shown in FIG. 2b, a tibia cutter 22 is mounted on the top end of tibia 12 using alignment device (not shown) and the top end of the tibia 12 is independently cut through a cutting groove 22-1. That is, in the conventional knee arthroplasty, the femur cutter 21 and tibia cutter 22 are mounted independently to the femur 11 and the tibia 12, then the femur 11 and tibia 12 are cut independently of each other. However, the conventional arthroplasty of cutting by aligning the femur 11 and the tibia 12 independently has a problem in that since in the actual surgery the femur 11 and the tibia 12 are not aligned in agreement to the mechanical axis, the cut surface of the femur bottom end and the cut surface of the tibia top end are not in parallel, so it is impossible to cut the extension gap accurately.

To make up for this drawback, the present applicant filed a patent application for an alignment and connection device 30 for connecting the alignment of femur cutter and tibia cutter and had it registered as Korean Patent No. 399489. As shown in FIG. 3, the alignment and connection device has a latching part 31 on the top and a pinhole part 32 on the bottom formed into a body by a side plate 33, and a fixation part has a plurality of pinholes 34 formed at a predetermined interval for pins P to be inserted.

Knee arthroplasty using the alignment and connection device according to the aforementioned patent will be briefly described with reference to FIG. 4 and FIG. 5. In such a replacement surgery, a femur cutter 21 is mounted on the bottom end of the femur 11 with pins, and then a latching part 31 of an alignment and connection device 30 is inserted into the cutting groove 21-1 of the femur cutter 21 and pins P are inserted into the pinholes of the alignment and connection device 30 for a tibia 12 to be driven. Thereafter, the alignment and connection device 30 is separated from the femur cutter 21 and pins P, and the tibia cutter 22 is mounted on the tibia 12 with the pins P that were driven into the tibia 12. Then, around the knee joint, the femur cutter 21 and the tibia cutter 22 are mounted in parallel each other on the bottom end of the femur 11 and the top end of the tibia 12, as shown in FIG. 5. Therefore, the cut surface of the femur bottom end and the cut surface of the tibia top end can be in parallel each other.

For reference, an extension gap is the gap between the cutting groove 21-1 of the femur cutter 21 and the cutting groove 22-1 of the tibia cutter 22 in FIG. 5. In other words, the extension gap is the sum of the cut length of the femur bottom end and the cut length of the tibia top end. In a normal surgery, the cut length of the femur bottom end is 8 mm and the cut length of the tibia top end is determined by the surgeon depending on the condition of the patient. Meanwhile, in surgery, since the position of the cutting groove 22-1 of the tibia cutter 22 is determined by the position of the pins P inserted into the pinholes 34 and driven into the tibia 12, the reference point of the extension gap becomes the position of the pinholes 34 of the alignment and connection device 30 for the pins P to be inserted. For example, if the surgeon determines the extension gap at 20 mm depending on the extent of damage of the patient's knee joint, the cut length of the femur bottom end becomes 8 mm and the cut length of the tibia top end becomes 12 mm. If you select any one of pinholes 34 that corresponds to the cut length of the tibia top end, and insert pin P, then eventually, the gap between the cutting groove 21-1 of the femur cutter 21 and the cutting groove 22-1 of the tibia cutter 22 becomes 20 mm.

Like this, the aforementioned patent uses the alignment and connection device 30 to make it possible for the cut surface of the bottom end of the femur 11 and the cut surface of the top end of the tibia 12 to be in parallel because the alignment of the femur cutter 21 and the tibia cutter 22 is not made independently of each other but in mutual connection.

However, the alignment and connection device 30 according to the aforementioned patent has a problem in that it is not possible to minutely follow the extension gap determined. As we have seen, the reference point of the extension gap is the position of the pinholes 34 of the alignment and connection device for pins P to be inserted, but these pinholes 34 are formed at an interval so it is not possible to follow the extension gap minutely and continuously. For example, if the surgeon determined the extension gap at 19.5 mm (the cut length of the femur bottom end: 8 mm, the cut length of the tibia top end: 11.5 mm) but there is no pinhole that corresponds to the extension gap, then the problem is you cannot determine the reference point of the extension gap required. Therefore, an alignment and connection device that can follow the determined extension gap minutely is required.

Another problem is that the alignment and connection device 30 according to the aforementioned patent cannot precisely compensate for the length of lax ligament if the ligament of knee joint becomes lax. For example, the surgeon determined the extension gap at 20 mm (the cut length of the femur bottom end: 8 mm, the cut length of the tibia top end: 12 mm). But in the case of the ligament being 0.5 mm lax, the extension gap selected is 19.5 mm (the cut length of the femur bottom end: 8 mm, the cut length of the tibia top end: 11.5 mm) to cut the femur and tibia, and finally an artificial knee joint of 20 mm (femur replacement: 8 mm, tibia replacement: 12 mm) is inserted to extend the ligament. Thus, the length of lax ligament can be compensated for. However, this device has a problem that the length of lax ligament cannot be precisely compensated for because pinholes 34 are formed at an interval as mentioned above. Therefore, in case the ligament of knee joint is lax, an alignment and connection device that can easily compensate for the lax ligament length is required.

In addition, according to the alignment and connection device 30 according to the above patent, the cut surface of the femur bottom end to be cut and the cut surface of the tibia top end to be cut are not aligned three-dimensionally. That is, the device has a problem in that it was possible to maintain parallel alignment of the femur 11 and the tibia 12 on the coronal axis, but it is not possible to maintain parallel alignment of the femur 11 and the tibia 12 on the sagittal axis and the rotation synchronization of the femur 11 and the tibia 12 on the transverse axis. For reference, parallel alignment on the coronal axis means the alignment on the mechanical axis of the femur and tibia when the knee joint is seen directly in front, as shown in FIG. 6. And parallel alignment on the sagittal axis means the alignment on the mechanical axis of the femur and tibia when the knee joint is seen laterally. And rotation synchronization on the transverse axis means the rotation synchronization of the tibia with respect to the femur when the knee joint is seen from top. Here, in the alignment and connection device 30 as shown in FIG. 5, it is possible to align the tibia in parallel with the femur with respect to the mechanical axis on the coronal axis, but on the sagittal axis, it is not possible to align the tibia in parallel with the femur with respect to the mechanical axis, so the tibia could be cut inclined on the sagittal axis. And it is not possible to synchronize the tibia with the rotation of the femur, so there is a fear that the tibia could be cut in rotation with respect to the femur. Therefore, an alignment and connection device by which it is possible to connect by three-dimensional alignment and synchronization of the femur cutter and the tibia cutter is required.

### SUMMARY OF THE INVENTION

It is an object of the present invention to solve the aforementioned problems by providing an alignment and connection device of femur cutter and tibia cutter by which it is possible to minutely follow the extension gap determined.

It is another object of the present invention to provide an alignment and connection device of femur cutter and tibia cutter by which it is possible to align the femur cutter and the tibia cutter three-dimensionally.

It is yet another object of the present invention to provide a method of knee arthroplasty using the alignment and connection device.

In accordance with the present invention, there is provided an alignment and connection device of femur cutter and tibia cutter comprising: a fixation part in which a guide slot is formed in front center and a through hole for a locking pin to be inserted is formed on the side of the guide slot and an indicating line is marked thereon; a supporting part which is assembled slidably to the guide slot of the fixation part, and having a slider marked with specifications corresponding to the extension gap determined and a lug portion inserted into the cutting groove of the femur cutter; and a fixing means for fixing the supporting part to the fixation part.

Further, in accordance with the present invention, there is provided a Method of knee arthroplasty comprising the steps of: determining the extension gap based on the extent of damage of the knee joint of the patient; adjusting the slider of the alignment and connection device based on the extension gap determined, then fixing the slider to the fixation part with a fixing means; mounting the tibia cutter on the bottom end of the femur and driving a selective rotation prevention pin into the rotation center point of the femur top end; inserting the lug portion of the alignment and connection device into the cutting groove of the femur cutter simultaneously with inserting the first and second selective rotation prevention holes of the alignment and connection device into the selective rotation prevention pin driven into the tibia; inserting the inclination identifying pin into the inclination identifying slot formed on the bottom of the fixation part of the alignment and connection device, then adjusting the inclination of the inclination identifying pin and the tibia axis; inserting a locking pin into the through hole formed on front side of the fixation part of the alignment and connection device and driving it into the tibia; separating the selective rotation prevention pin and the alignment and connection device respectively from the tibia and femur cutter; mounting a tibia cutter with the locking pin driven into the tibia; cutting the femur bottom end through the femur cutter mounted on the femur, bending the femur in a bended state with respect to the tibia, and inserting again the separated alignment and connection device onto the locking pin driven into the tibia; positioning a cutting block size determination device in the femur such that the guide is placed on the top of the lug portion of the alignment and connection device and the bent part is placed on the first anterior cortex of the femur cut in advance; adjusting the cutting block size determination device to determine the size of the cutting block; cutting with the determined cutting block the posterior condyle, posterior chamfer, anterior chamfer and anterior cortex of the femur; and replacing an artificial knee joint between femur and tibia after cutting the top end of tibia by tibia cutter.

### BRIEF DESCRIPTION OF THE DRAWINGS

Other objects and aspects of the present invention will become apparent from the following description of embodiments with reference to the accompanying drawings in which:

FIG. 1 is a view illustrating an ideal bone cutting of extension gap;

FIG. 2a is a view for describing mounting a femur cutter on the bottom end of the femur using a conventional alignment device;

FIG. 2b is a view for describing mounting a tibia cutter on the top end of the tibia using a conventional alignment device;

FIG. 3 is a perspective view of a conventional alignment and connection device;

FIGS. 4 and 5 are drawings for describing a knee arthropolasy using the conventional alignment and connection device shown in FIG. 3;

FIG. 6 is a view for describing a coronal axis, sagittal axis, transverse axis, and mechanical axis;

FIG. 7 is a perspective view showing an assembled state of an alignment and connection device according to the present invention;

FIG. 8 is a perspective showing a disassembled state of an alignment and connection device according to the present invention;

FIG. 9 is a cross-section taken along line A-A′ of FIG. 7;

FIG. 10 is a view showing how an alignment and connection device according to the present invention is mounted;

FIG. 11 is a view showing how an alignment and connection device and a cutting block size determination device according to the present invention are mounted on a tibia and femur respectively;

FIG. 12 is a view showing how a cutting block is mounted on a femur.

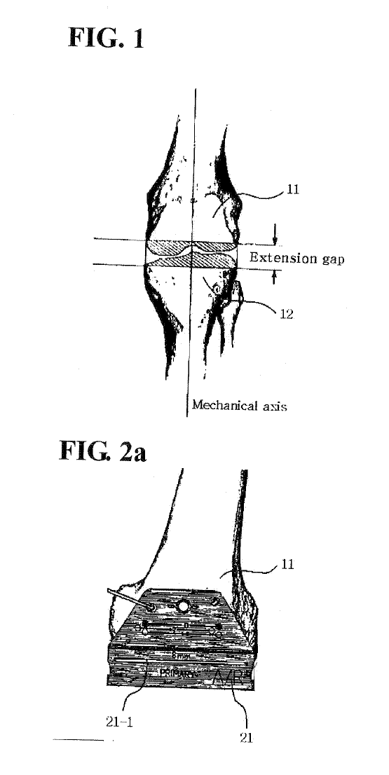

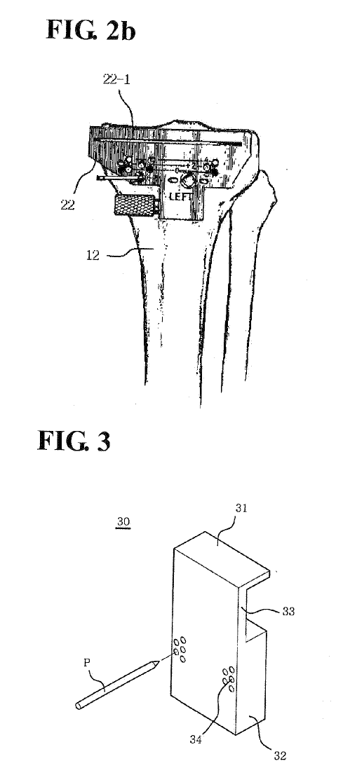

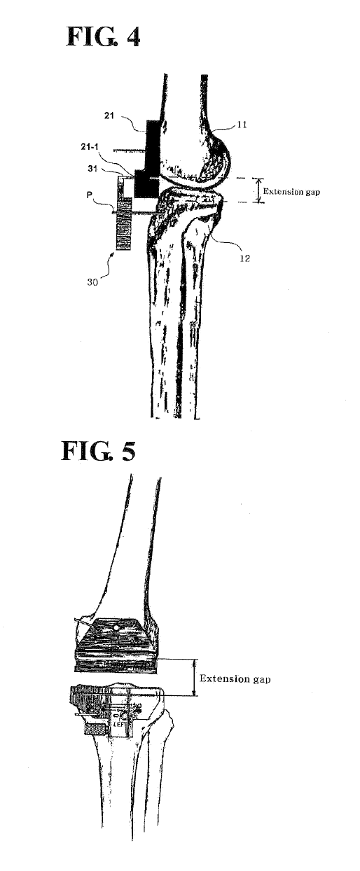

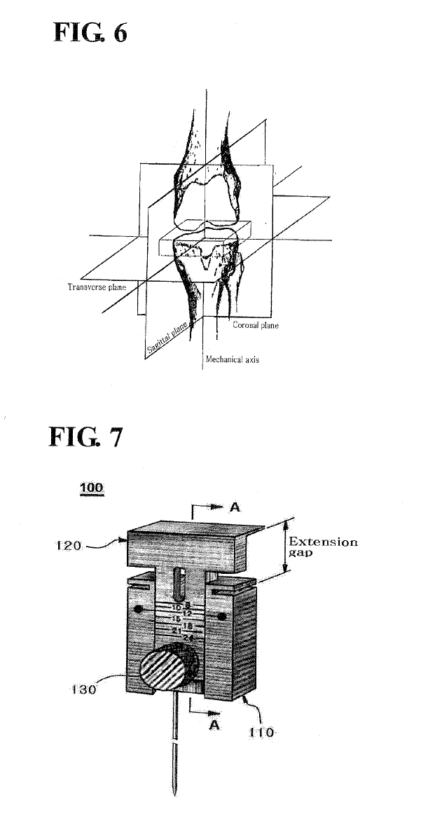

### DETAILED DESCRIPTION OF THE INVENTION

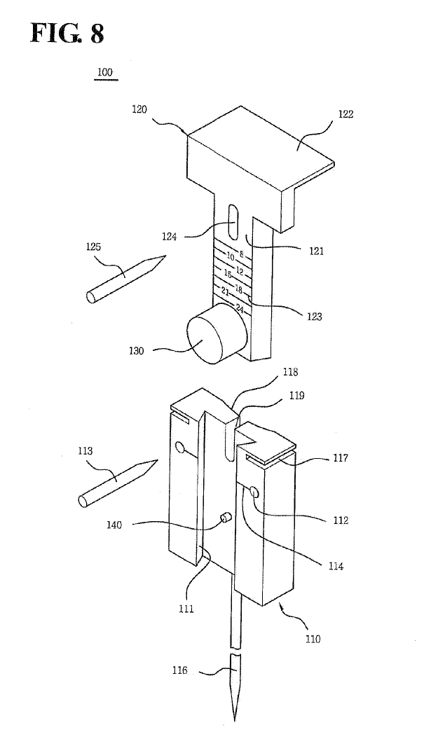

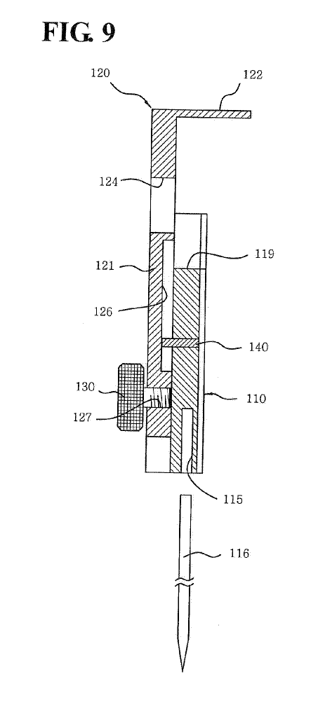

The present invention will be described in detail with reference to the accompanying drawings. FIG. 7 is a perspective view showing an assembled state of an alignment and connection device according to the present invention, and FIG. 8 is a perspective view showing a disassembled state of the alignment and connection device according to the present invention, and FIG. 9 is a cross-section taken along line A-A′ of FIG. 7. For the sake of convenience, the same symbols are given to the constituents that are the same with the conventional device.

Referring to FIGS. 7 through 9, an alignment and connection device 100 according to the present invention includes a fixation part 110, supporting part 120 and a fixing means 130.

The fixation part 110 includes a guide slot 111 formed in front center. To the guide slot 111 is slidably assembled a slider 121 of the supporting part 120 which will be described later. On both sides of the guide slot 111 are formed a pair of through holes 112, and into the through holes 112 are inserted locking pins 113 before driven into the top end of tibia 12. And on both sides of the front of the fixation part 110 are marked indicating lines 114 for indicating the specifications marked on the slider 121. It is preferable that an inclination identifying slot 115 for an inclination identifying pin 116 to be inserted is formed in a predetermined length on the bottom of the fixation part 110, in order to be able to identify whether the sagittal axis is inclined. It is also preferable that an extension gap preview slot 117 is formed above the through hole 112 of the fixation part 110 so as to preview the extension gap. Also, part of the rear side of the fixation part is preferably in a predetermined round 118 form so as to come into close contact with the front protrusion of the tibia.

The supporting part 120 is slidably assembled to the guide slot 111 of the fixation part 110, and the upper end of the supporting part 120 is configured so as to be inserted in the cutting groove 21-1 of the femur cutter. Specifically, the supporting part 120 comprises the slider 121 that is slidably assembled to the guide slot 111 of the fixation part 110 and a lug portion 122 that is inserted into the cutting groove 21-2 of the femur cutter 21. Also, on the front of the slider 121 are marked specifications 123 corresponding to the extension gap determined.

It is preferable that the contact surface of the slider 121 and the guide slot 111 is in an inwardly tapered form, but it is not limited to this, and a stepped form is also possible. Furthermore, on the upper side of the slider 121 is formed a first selective rotation prevention hole 124, and on the upper side of the guide slot 111 of the fixation part 110 is formed a second selective rotation prevention hole 119 that corresponds to the first selective rotation prevention hole 124. Here, the first and second selective rotation prevention holes 124 and 119 are provided lengthwise in a long hole, and the top end of the second selective rotation prevention hole 119 is formed in an open end as shown in drawing. The first and second selective rotation prevention holes 124 and 119 are provided to be inserted onto a selective pin 125 that was driven in advance in the rotation center of the tibia top end, as will be described later. The diameter of the selective rotation prevention pin 125 is formed substantially equal to or less than the width of the first and second selective rotation prevention holes 124 and 119.

Meanwhile, as shown in FIG. 9, a longitudinal slot 126 is formed on the rear side of the slider 121, and a protruding pin 140 introduced into the long slot 126 can be forcibly inserted into the fixation part 110 from the back. Due to the forcibly inserted protruding pin 140, the slider 126 and the fixation part 110 can be prevented from breaking away each other.

It is preferable that the fixing means 130 is a locking bolt to be inserted into the screw hole 127 of the slider 121.

The operation of the alignment and connection device according to the present invention will be described together with the description of the method of knee arthroplasty.

In the method of knee arthroplasty using the alignment and connection device according to the present invention, first the slider 121 is adjusted to fit the extension gap determined by the surgeon, and then the slider 121 is fixed to the fixation part 110 with the locking bolt 130. That is, as shown in FIG. 7, if the surgeon determined the extension gap at 20 mm (the cut length of the femur bottom end: 8 mm, the cut length of the tibia top end: 12 mm) according to the condition of the patient, the slider 130 is fixed to the fixation part 110 with the locking bolt 130 such that the indicating line of the fixation part indicates the graduation 12 of the slider 121. Then, the length from the actual extension gap preview slot 117 to the lug portion 122 becomes 20 mm, the extension gap. That is, the graduation listed on the slider 121 shows the cut length of the tibia top end to be cut. At this time, the extension gap is the length of the lug portion 122 from the preview slot 117. However, the present invention is not limited to this, but may as well be designed such that the graduation itself listed on the slider 121 shows the extension gap, which is the sum of the cut length of the femur bottom end and the cut length of the tibia top end.

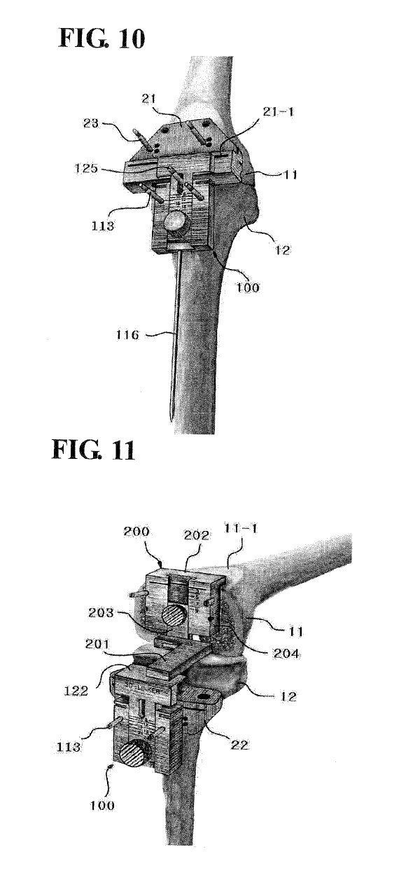

When the slider 121 is fixed according to the extension gap, the femur cutter 21 is mounted on the bottom end of the femur 11 with the pin 23 and the selective rotation prevention 125 is driven into the rotation center point of the top end of the tibia 12, as shown in FIG. 10. Then, the lug portion 122 of the alignment and connection device 100, and the first and second selective rotation prevention holes 124 and 119, are inserted and left into the cutting groove 21-1 of the femur cutter 21, and the selective rotation prevention pin 125 driven into the tibia 12, respectively. At this time, since the diameter of the selective rotation prevention pin 125 is substantially equal to or less than the width of the first and second selective rotation prevention holes 124 and 119, and the selective rotation prevention pin 125 is driven into the rotation center of the tibia top end, the alignment and connection device 100 is aligned while maintaining the rotation synchronized on the transverse axis with respect to the femur cutter 21.

Thereafter, the inclination identifying pin 116 is inserted into the inclination identifying slot 115 formed on the bottom of the fixation part 110, and the extent of agreement of the inclination identifying pin 116 and the tibia axis is checked. If the inclination identifying pin 116 and the tibia axis are inclined at a predetermined angle (that is, if the alignment on the sagittal axis is not agreed), the tibia 12 is moved to make the tibia axis agree with the inclination identifying pin 116. At this time, because the selective rotation prevention pin 125 driven into the tibia can be moved up and down within the selective rotation prevention hole 124 with the rotation center point of the tibia as the hinge point, the alignment and connection device can be maintained in parallel arrangement also on the sagittal axis with respect to the femur cutter 21.

Meanwhile, parallel arrangement on the coronal axis also can be accomplished by minutely moving the tibia left or right of the inclination identifying pin 116.

Like this, when alignment with respect to the three axes (coronal axis, sagittal axis and transverse axis) is accomplished, the locking pin 113 is inserted into the through hole 112 formed on front side of the fixation part 110 while maintaining the alignment and is driven into the tibia 12. Because the alignment and connection device 100 has a 3-axis alignment accomplished, the locking pin 113 driven into the tibia 12 has also a 3-axis alignment accomplished. Drive the locking pin 113 into the tibia and separate the selective rotation prevention pin 125 and the alignment and connection device 100 from the tibia 12 and the femur cutter 21, respectively. Then, only two locking pins 113 will remain. Thereafter, mount the tibia cutter 22 with the locking pin 113 remaining in the tibial 12. Then, as shown in FIG. 5, the femur 11 will have the femur cutter 21 mounted, and the tibia 12 will have only the tibia cutter 22 mounted. Although not shown, the three-dimensional shape of FIG. 5 shows alignment accomplished three-dimensionally. Also, the position of the cutting groove 22-1 of the tibia cutter 22 comes into position corresponding to the position of the preview hole 117 of the alignment and connection device 100.

As mentioned above, the alignment and connection device according to the present invention has the following advantages.

First, it can provide an alignment and connection device that can minutely follow the extension gap determined. That is, in the conventional alignment and connection device, the extension gap is followed by pinholes with intervals, so it was not possible to follow the extension gap minutely, but in the alignment and connection device according to the present invention, the extension gap is followed by the slider and the sliding method of the fixation part, so it has an effect that the extension gap can be followed linearly and continuously.

Second, if the ligament of knee joint becomes lax, the length of lax ligament can be easily compensated for. As mentioned above, the alignment and connection device according to the present invention can follow the extension gap continuously, so it has an effect that surgery is possible by easily changing the extension gap as much as the length of ligament to be compensated for.

Third, it can provide an alignment and connection device that can connect by three-dimensional synchronization of the alignment of the femur cutter and tibia cutter. That is, the present invention can make rotation synchronization on the transverse axis by the selective rotation prevention pin, and can accomplish parallel alignment on the sagittal axis and minute parallel alignment on the coronal axis by the inclination identifying pin, so it has an effect that connection is possible by three-dimensional synchronization of the alignment of the femur cutter and tibia cutter.

After the femur cutter 21 and the tibia cutter 22 are mounted on the femur 11 and the tibia 12 respectively, the alignment and connection device 100 according to the present invention and the cutting block size determination device according to the PCT Application No. PCT/KR2004/000703 filed by the present applicant are used in surgery of knee arthroplasty, which will be described below. Note: The cutting block size determination device and circumferential explanation on this are described in detail in PCT Application No. PCT/KR2004/000703 which is herein incorporated by reference.

After the femur cutter 21 and the tibia cutter 22 are mounted respectively on the femur 11 and the tibia 12, the bottom end of the femur 11 is cut by the femur cutter 21. As shown in FIG. 11, after the femur 11 is bent in a bended state with respect to the tibia 12, the removed alignment and connection device 100 is inserted again onto the locking pin 113 driven into the tibia 12. Next, the cutting block size determination device 200 disclosed in PCT Application No. PCT/KR2004/000703 is positioned in the femur 11 with the primary anterior cortex 11-1 cut in advance to determine the cutting block size.

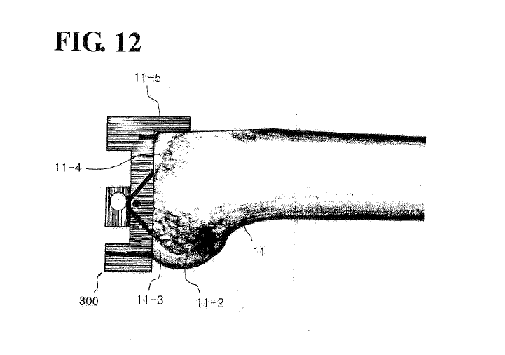

Here, in the cutting block size determination device 200, make a guide 201 be placed on the top of the lug portion 122 of the alignment and connection device and a bent part 202 be placed on the primary anterior cortex 11-1 cut in advance. When the cutting block size determination device 200 is positioned in the tibia 11 like this, it judges the graduation of a body 204 indicated by the indicating line of the slider 203 to determine the cutting block size. Next, as shown in FIG. 12, the posterior condyle 11-2, the posterior chamfer 11-3, the anterior chamfer 11-4 and the anterior cortex 11-5 of the femur are cut with the cutting block 300 of determined size.

Thereafter, the top end of the tibia 12 is cut by the tibia cutter 22, and an artificial knee joint is replaced between the cut femur and tibia.

As described above, according to the alignment and connection device of the present invention, there is provided an alignment and connection device by which it is possible to follow the determined extension gap minutely, compensate for the length of lax ligament easily, and connect by three-dimensionally synchronizing the alignment of femur cutter and tibia cutter. In addition, the knee arthroplasty using such an alignment and connection device has effects of preventing side effects after surgery and prolonging the life of the replaced artificial knee joint.

While the present invention has been described with reference to exemplary embodiments thereof, it will be understood by those skilled in the art that various changes in form and details may be made therein without departing from the scope of the present invention as defined by the following claims.

What is claimed is:

1. An alignment and connection device of femur cutter and tibia cutter comprising:
a fixation part in which a guide slot is formed in front center and a through hole for a locking pin to be inserted is formed on the side of the guide slot and an indicating line is marked thereon;
a supporting part which is assembled slidably to the guide slot of the fixation part, and having a slider marked with specifications corresponding to the extension gap determined and a lug portion inserted into the cutting groove of the femur cutter; and
a fixing means for fixing the supporting part to the fixation part.

2. The alignment and connection device of claim 1, wherein a first selective rotation prevention hole for a selective rotation prevention pin to be inserted is formed on the slider, and a second selective rotation prevention hole corresponding to the first selective rotation prevention hole is formed on the guide slot of the fixation part.

3. The alignment and connection device of claim 2, wherein the diameter of the selective rotation prevention pin is substantially equal to or less than the width of the first and second selective rotation prevention holes.

4. The alignment and connection device of claim 1, wherein an inclination identifying slot for an inclination identifying pin to be inserted so as to identify whether the sagittal axis is inclined or not is formed on the bottom of the fixation part in a predetermined length.

5. The alignment and connection device of claim 1, wherein a longitudinal slot is formed on the rear side of the slider and a protruding pin introduced into the long slot is forcibly inserted from the back in the fixation part.

6. The alignment and connection device of claim 1, wherein an extension gap preview slot for previewing the extension gap is formed on the through hole of the fixation part is formed.

7. Method of knee arthroplasty comprising the steps of:
determining the extension gap based on the extent of damage of the knee joint of the patient;
adjusting the slider of the alignment and connection device based on the extension gap determined, then fixing the slider to the fixation part with a fixing means;
mounting the tibia cutter on the bottom end of the femur and driving a selective rotation prevention pin into the rotation center point of the femur top end;
inserting the lug portion of the alignment and connection device into the cutting groove of the femur cutter simultaneously with inserting the first and second selective rotation prevention holes of the alignment and connection device into the selective rotation prevention pin driven into the tibia;
inserting the inclination identifying pin into the inclination identifying slot formed on the bottom of the fixation part of the alignment and connection device, then adjusting the inclination of the inclination identifying pin and the tibia axis;
inserting a locking pin into the through hole formed on front side of the fixation part of the alignment and connection device and driving it into the tibia;
separating the selective rotation prevention pin and the alignment and connection device respectively from the tibia and femur cutter;
mounting a tibia cutter with the locking pin driven into the tibia;
cutting the femur bottom end through the femur cutter mounted on the femur, bending the femur in a bended state with respect to the tibia, and inserting again the separated alignment and connection device onto the locking pin driven into the tibia;
positioning a cutting block size determination device in the femur such that the guide is placed on the top of the lug portion of the alignment and connection device and the bent part is placed on the first anterior cortex of the femur cut in advance;
adjusting the cutting block size determination device to determine the size of the cutting block;
cutting with the determined cutting block the posterior condyle, posterior chamfer, anterior chamfer and anterior cortex of the femur; and
replacing an artificial knee joint between femur and tibia after cutting the top end of tibia by tibia cutter.
